(19) United States
(12) Patent Application Publication
(10) Pub. No.: US 2020/0179148 A1
(43) Pub. Date: Jun. 11, 2020

(54) Medical Device for Gastric Reductions

(71) Applicant: Aspire Surgical Pty Ltd, Ormond (AU)

(72) Inventors: Christopher Thick, Carnegie (AU); Timothy Farrant, Carnegie (AU)

(21) Appl. No.: 16/349,529

(22) PCT Filed: Nov. 14, 2017
(86) PCT No.: PCT/AU2017/051247

§ 371 Date: May 13, 2019

(30) Foreign Application Priority Data
Nov. 14, 2016 (AU) 2016904632

(51) Int. Cl. A61F 5/00; A61B 17/12

(52) U.S. Cl. CPC A61F 5/0066; A61F 5/0063; A61B 17/12009

(57) ABSTRACT

The present invention relates to a medical device that is for medical procedures relating to gastric reductions. The device has an elongate body having a 5 flexible portion and a stiffened portion extending from the flexible portion, the stiffened portion can be gripped by a grasper for manoeuvring the device about a gastric conduit of a patient, and a locking mechanism that is operable to secure the flexible portion in a looped configuration.

### FIELD OF THE INVENTION

The present invention relates to a medical device that can be used is for some medical procedures relating to gastric reductions.

### BACKGROUND OF THE INVENTION

Surgical procedures for gastric reductions are an effective means for managing weight loss in overweight patients including obese and morbidly obese patients. Some of the benefits that can result from gastric reductions include reducing blood pressure, reducing or delaying the onset of diabetes, reducing or delaying the onset of heart disease and improve self-esteem.

There are many different surgical procedures that result in gastric reductions and weight loss including, but not limited to, gastric bypass procedures and sleeve-gastrectomy. In both types of procedures, a pouch having a volume smaller than the patient's stomach is formed and the stomach is either resected or bypassed which involves anastomosis at various positions of the stomach and small intestine. The device used in these procedures may be located around the pouch with the aim to protect against dilatation of the pouch, and to protect areas of anastomosis. Moreover, the device can assist in regulating the volumetric flow of food into the digestive system which can provide lasting weight reduction.

### SUMMARY OF THE INVENTION

An embodiment relates to a medical device for gastric reductions, the medical device includes:
an elongate body having a flexible portion and a stiffened portion extending from the flexible portion, the stiffened portion can be gripped by a grasper and thereby manoeuvre the stiffened portion between internal organs of a patient to place the device about a gastric conduit of a patient;
a locking mechanism that is operable to secure the flexible portion in a looped configuration at a selectable diameter about the gastric conduit, the locking mechanism having: i) an aperture in the flexible portion that is positioned adjacently to the stiffened portion, and ii) a plurality of locking teeth that form part of the flexible portion that are positioned remotely from the stiffened portion;
wherein the flexible portion includes a free end that can be advanced through the aperture until one of the locking teeth engages the aperture and secures the looped configuration in the desired diameter.

An advantage of this embodiment is that the stiffened portion can be held by a clinician with forceps or graspers, which provides an ability to hold and manoeuvrer the device into the desired position, and in addition whilst being held, the free end of the flexible portion can be advanced through the aperture.

The stiffened portion can be severed from the device at any time during the procedure, typically after the flexible portion has been secured in a looped configuration about the gastric conduit and the size of the looped configuration set.

When the device is arranged in the looped configuration, the body of the device will have an inwardly facing surface that can, depending on the profile of the inwardly facing surface forms an inner perimeter of the device and an outwardly facing surface that can, depending on the profile of the outwardly facing surface form part of an outer perimeter of the device. In addition, when in the looped configuration the medical device can be used as a ring about a gastric conduit in a medical procedure.

Surgical graspers or forceps have a pair of jaws at the distal end of a stem and a handle at a proximal end, including finger operated levers that operate the jaws. Depending on the particular grasper(s) being used, one of the jaws may be moveable toward the other, or alternatively, both of the jaws may be movable toward and away from each other in a scissor action. In addition, the jaws can be pivotally connected about a common axis and when the jaws are being moved from an opened position to a closed position, the jaws approach each other at a reducing angle and typically reach a parallel position when fully closed. The jaws may also be pivoted about spaced axes and in this situation, when the jaws are being moved from an opened position to a closed position, the jaws approach each other at a reducing angle and can be parallel in a spaced apart position, for example, when the jaws are fully closed.

In one embodiment, the grasping section may align with or protrude from an adjacent section of the body of the device.

In an embodiment, the device has a grasping section having a gripping surface for jaws of a grasper, the gripping surface being adapted to receive the jaws of a grasper by being recessed to an outer face of the body.

In an embodiment, the recessed area extends about at least part of the perimeter of the stiffened portion, and the grasping section includes an at least one abutment surface that extends laterally to the gripping surfaces, jaws of the grasper or forceps can butt against the abutment surface.

The abutment surfaces can provide an advantage of preventing the graspers or forceps from twisting when engaging the gripping surface, or provide a reaction surface when the device is being either pulled or pushed.

The grasping section has two abutment surfaces extending outwardly at opposite ends of the recessed area.

The grasping section may be provided in at least one of, and possibly both of, the stiffened portion or the flexible portion.

Ideally, the grasping section is located in the stiffened portion only.

In one embodiment, the abutment surfaces may extend outward at opposite ends of the recessed area.

The recessed area of the grasping section may be inward of a line extending tangentially of the outer face of the body of the device. An advantage of the grasping section having the recessed area is that the sides of the jaws of the graspers can engage the abutment surfaces as a clinician manoeuvres the device. Another advantage is that the recessed area of the grasping section is less likely to catch or tear tissue as the device is being manoeuvred into position by a clinician.

In one example, the grasping section may be located in the stiffened portion. An advantage this provides is that the stiffened portion can be more easily grasped and manoeuvred through connective tissue and into position by the clinician, compared to grasping a flexible section of the body of the device.

The grasping section may have any suitable length for grasping with forceps or graspers, and ideally the grasping section has a length in the range of 8 to 18 mm, and suitably in the range of 10 to 16 mm, and even more suitably in the range of 10 to 13 mm.

The gripping surfaces of the grasping section may have flat planar surfaces on which the jaws of a grasper can contact when closing.

The grasping section may have flat planar surfaces arranged about at least part of the perimeter of the recessed area of the stiffened portion.

The grasping section may have a first pair of gripping surfaces that face outwardly from opposite sides of the body of the device.

For example, the grasping section may have a first pair of gripping surfaces such that when the device is arranged in a looped configuration, the first pair of gripping surfaces include a first gripping surface that aligns with an inwardly face surface of the body and a second gripping surface that aligns with an outwardly facing surface of the body.

Ideally the first and second gripping surfaces are planar surfaces.

An advantage of the first and second gripping surfaces is that they can be readily gripped using surgical graspers or forceps that have jaws that are spaced apart in a parallel relationship when closing.

Ideally, the thickness of the body of the device between the first and second gripping surfaces is in the range of 2.0 to 5.0 mm. More suitably, the thickness of the device between the first and second gripping surfaces is in the range of 3.0 to 4.0 mm.

The gripping section may also include at least one second pair of gripping surfaces that are arranged at an angle to the first pair of planar surfaces. The second pair of gripping surfaces may be oppositely disposed to each other about the body of the device and face outwardly of the body of the device.

The recessed area of the grasping section may have a smaller cross-section than an adjacent section of the stiffened portion. Ideally, the adjacent larger section of the grasping section is outside of the recessed area.

The elongated body of the device may have a width, excluding a very tip portion of the stiffened portion, in the range of 2 mm to 10 mm. Ideally the elongated body has a widest point in the range of 6 to 10 mm, and ideally approximately 7 mm. The width of the device is a dimension laterally sideways to a longitudinal axis of the body.

In one embodiment, the second pair of gripping surfaces may include side surfaces that are arranged at an angle to the first gripping surface.

The second pair of gripping surfaces may be flat planar surfaces.

The second pair of gripping surfaces may include side planar surfaces arranged at an angle to the first pair of gripping surfaces. An advantage of the side planar surfaces is that they are ideally arranged to be engaged by jaws of a grasper in which the jaws close together in a scissor action at an angle to each other, and therefore can more easily be seated on a flat part of the side planar surfaces.

The device may include at least one suture opening for suturing the device in an operative position when implanted in a patient. The suture opening may be arranged within the elongate body of the flexible portion. In other words, the suture opening does not ideally protrude beyond the walls of the flexible portion and, therefore, are unlikely to cause tearing of tissue whilst the device is being manoeuvred into position.

In other words, the hazards associated with suture rings protruding from the elongate body can be lessened.

The or each suture opening may be defined between an inner wall and an outer wall of the elongate body.

The outer wall may have a height that is less than the inner wall. The height of the outer wall is, ideally, in a direction extending between the inwardly facing surface and the outwardly facing surface. An advantage this provides is that is allows for easier suturing and for tissue in-growth, which helps to secure the device in place after the sutures have dissolved or broken down.

In one embodiment, the or each suture opening may be arrange at a widest section of the device and the outer wall defining the or each suture opening may protrude outwardly of a main outside face of the elongated body.

The outer wall may have a height half the height of the inner wall. For example, the outer wall may have a height in the range of 1.5 to 2.5 mm and ideally, approximately 2.0 mm

In one embodiment, the or each suture opening may be located within the flexible portion of the device.

The outer wall of the or each suture opening includes a flexible part that can be flexed relative to the elongated body. The flexible part may be provided by the entire outer wall.

In one embodiment, the outer wall of the or each suture opening includes a flexible part, and when in a relaxed state the flexible part protrudes outwardly of the elongated body and when the flexible part is placed in tension, the outer wall can move inwardly.

In another embodiment, the outer wall of the or each suture opening may be a flexible wall partition, and when the flexible wall portion of the device is placed in tension, for example, when subjected to an external force while being manoeuvred into an operative position, the flexible wall partition can flex inwardly.

For example, the outer wall of the or each suture opening may move inwardly, from an outer position in which the outer wall is disposed outside of the elongate body to a position substantially in alignment with the elongate body when the device is tensioned longitudinally.

In one embodiment, the suture opening may be arranged at a wide section of the elongated body.

The or each suture opening is located adjacent to the aperture for receiving the free end.

The or each suture opening may be elongated. For example, the suture opening(s) may have a width ranging from 0.5 to 2.0 mm and, ideally, from 0.5 to 1.5 mm. These dimensions of the or each suture openings is measured when the outer wall of the suture opening substantially aligns with the elongate body. The suture opening may also have a length ranging from 5.0 to 15.0 mm, and ideally in the range from 8.0 to 12 mm, and even more suitably approximately 11.5 mm. The suture openings can allow for tissue in growth, which helps to secure the device in place after the sutures have dissolved or broken down.

The device may comprise two suture openings located side-by-side in the elongated body of the device.

Each suture opening may be arranged in the flexible portion that forms part of the looped configuration. Ideally, the suture openings may be located adjacent to the aperture of the locking mechanism.

The free end of the device may have a reduced cross-section portion that can be threaded through the aperture without frictionally engaging the aperture. The reduced cross-section portion may be located at a distal most end of the device.

The free end also includes an engaging section having an enlarged cross-section portion extending from the reduced cross-section portion, the engaging section having an enlarged cross-section that is sized to frictionally engage the aperture.

The reduced cross-section portion may have a length that is ideally at least twice the thickness of the elongated body at the aperture. The advantage this provides is that the reduced cross-section can be threaded through the aperture, and protrude from the aperture without frictional engaging the aperture.

For example, the reduced cross-section portion may have a length of at least 10 mm, and suitably in the range of 10 to 20 mm, and even more ideally in the range of 12 to 15 mm.

The leading portion may have a flattened profile that can be engaged by jaws of a grasper and has ridges extending transversely to a longitudinal axis of the device to assist in the jaws gripping onto the leading section.

The reduced cross-sectional portion may have a flattened profile that can be engaged by jaws of a grasper and has ridges extending transversely to a longitudinal axis of the device.

The ridges of the leading portion or the reduced cross-sectional portion can be grasped by the grasper or forceps to assist manoeuvring the device into position.

It may be possible to frictionally retain the engaging section in the aperture without a clinician manually holding the device. For example, the engaging section may be able to hold the device in a preliminary looped configuration, prior to engagement of the locking teeth and adjustment of the looped configuration into a desired diameter.

The engaging section may have a notch which when pulled through the aperture can engage the aperture to retain the device in the preliminary looped configuration. The notch may provide a localised region of increased friction between the engaging section and the aperture. Ideally the notch does not extend along the device by a length greater than the depth of the aperture.

The stiffened portion may have a curved tip at an end thereof. The curved tip may assist in threading the stiffened portion between the organs of a patient.

The device can be manoeuvred between internal organs of a patient by grasping either one or both of the stiffened portion or the flexible portion. In addition, the leading section of the device can be used as a forward end of the device, as viewed in the direction of movement of the device whilst being manoeuvred into position. Alternatively, the stiffened section of the device can be used as a forward end if the device, as viewed in the direction of movement of the device whilst being manoeuvred into position. A clinician can use either end of the device depending on the situation and their own preference, with insignificant risk to damage the patient's blood vessels or other tissue. In other words, one advantage of the device is that the elongate body is free of protrusions or projections, thereby allowing the device to be manoeuvred into position using either the stiffened section or the leading section as the forward end.

At least part of the stiffened portion may be integrally formed with the flexible portion. For example, the stiffened portion may include a web that is integrally formed with the flexible portion. The web may be centrally located in the stiffened portion and extend diametrically across the stiffened portion.

The stiffened portion may also include an outer rigid portion, for example, an outer sleeve or sock arranged over the central portion. The outer rigid portion may also be arranged on opposite flanks of the stiffened portion. The outer rigid portion may include a pair of flanking members that are interconnected by lugs. The lugs may extend through the central web.

The flexible portion may have a substantially uniform flexibility along its length. An advantage this can provide is that when the device is arranged in the looped configuration, the looped configuration forms a ring and suitably, a substantially circular ring.

In one embodiment, the flexible portion can form a substantially circular ring when one of the locking teeth is engaged to secure the device in a closed loop configuration.

In another embodiment, the flexible portion can form a tear drop shape when one of the locking teeth is engaged to secure the device in a closed loop configuration

The flexible portion may have any suitable cross-section including a squared cross-section, an oval cross-section, a rounded cross-section and so forth. Suitably however, the flexible portion includes an asymmetric cross-section, such as a hemispherical cross-section or an arched cross-section.

The flexible portion has a shaped cross-section in which an inwardly facing surface of the elongated body has a flattened profile and the outwardly facing surface of the elongate body has a curved profile.

Ideally, the shaped cross-section of the flexible portion extends along the entire length of flexible portion between the aperture to locking teeth.

Throughout this specification, the cross-section of the flexible portion and the stiffened portion, or parts thereof, refers to a cross-section measured laterally to a longitudinal axis of the flexible portion.

The aperture of the locking mechanism is disposed adjacent to the stiffened portion, which in other words means that the aperture is locate toward the stiffened portion.

The locking teeth of the flexible portion are separated by neck sections.

The neck sections may have a reduced cross-section compared to the cross-section of the locking teeth.

Ideally, the neck sections and the locking teeth both have the same or a similar width at an inwardly facing surface of the device.

The neck sections may be recessed inward of the outer surface of the locking teeth when in the looped configuration. An advantage in the locking teeth and the neck sections having a similar or the same width at the inner surface is that a clinician may adjust the diameter of the looped configuration by releasing outer edges of the locking teeth through the aperture. This may, for example, be achieved by temporarily deforming the aperture and/or the locking teeth.

Ideally, the neck sections and the locking teeth both extend from an inner diameter of the flexible portion, and have a substantially equal dimension, namely the same width, at the inner diameter of the locking teeth. Suitably, the locking teeth extend above the neck portion in a direction from the inner perimeter to the outer perimeter.

In one embodiment, the neck sections and the locking teeth extend from an inner diameter or inner surface of the body of the device, and the width of the neck sections is at least 50% of the width of the locking teeth at the inner diameter. Suitably, the width of the neck sections is at least 75% of the width of the locking teeth at the inner diameter or inner surface of the body of the device. Even more suitably, the width of the neck sections is at least 90% of the width of the locking teeth at the inner diameter or inner surface of the body of the device. Still even more suitably, the width of the neck sections is approximately 100% of the width of the locking teeth at the inner diameter or inner surface of the body of the device.

The locking teeth may be angled to a longitudinal axis of the body of the device. For example, the locking teeth may angle away from the free end of the device. The locking teeth may form an acute angle between a longitudinal axis of the device in a direction toward the stiffened end of the device.

The aperture may have a first opening on the inner surface (diameter) of the device, when in a looped configuration, that is larger than a second opening on the outer (diameter) of the device. An advantage this provides is that the locking teeth can be more easily manoeuvred in a direction through the aperture that reduces the size of the looped configuration.

The length of the neck sections, in the direction of the longitudinal axis of the device, is approximately equal to the dimension of the device in a direction along the aperture. In other words the length of the neck sections is approximately equal to the thickness of the body of the device at the aperture. An advantage this provides that there will be little relative movement between the locking teeth and the aperture of the device when the device is locked in a looped configuration.

An embodiment relates to a medical device for gastric reductions, the medical device includes:
an elongate body having a flexible portion and a stiffened portion extending from the flexible portion, the stiffened portion having a gripping section that has at least one pair of flat surfaces that face outwardly from opposite sides of the gripping section and that can be gripped by a grasper and thereby manoeuvre the stiffened portion between internal organs of a patient to place the device about a gastric conduit of a patient;
a locking mechanism that is operable to secure the flexible portion in a looped configuration at a selectable diameter about the gastric conduit, the locking mechanism having: i) an aperture in the flexible portion that is positioned adjacently to the stiffened portion, and ii) a plurality of locking teeth that form part of the flexible portion that are positioned remotely from the stiffened portion;
wherein the flexible portion includes a free end, having a reduced cross-section portion that can be threaded through the aperture without frictionally engaging the aperture, and the reduced cross-section portion has a flat profile that can be gripped by graspers and can be advanced through the aperture until one of the locking teeth engages the aperture and secures the looped configuration in the desired diameter.

### BRIEF DESCRIPTION OF THE DRAWINGS

A preferred embodiment will now be described with reference to the accompanying drawings, of which:

FIG. 1 is a perspective view of a medical device having an elongate body having a stiffened portion and a flexible portion, and in which the elongated body is located in a linear arrangement ready for use in a medical procedure, an upwardly facing surface of the device in FIG. 1 that can form the inwardly facing surface or inner perimeter when in a looped configuration, and the downwardly facing surface of the device in FIG. 1 forms an outwardly facing surface that can at least form part of an outer perimeter when arranged in a looped configuration;

FIG. 2 is a side view of the medical device shown in FIG. 1;

FIGS. 2A, 2B and 2C are sectional views through the cross-sections A-A, B-B and C-C respectively in FIG. 2;

FIG. 3 is an enlarged view of a grasping section of the elongated body in FIG. 1;

FIG. 4 is a plan view of the device shown in FIG. 1 showing the inwardly facing surface of the device, in which outer walls of the suture openings are curved outward;

FIG. 5 is an enlarged side view of a region of the flexible portion adjacent to the stiffened portion of the device shown in FIG. 2;

FIG. 6 is an enlarged plan view of region of the device shown in FIG. 5, in which outer wall of the suture openings are located in alignment with the elongate body;

FIG. 7 is a perspective view of a central web of the stiffened portion that can be used as a trocar for locating the device in position; and

FIG. 8 is a perspective view of a pair of outer rigid members that are fitted to either side of the central web shown in FIG. 7;

FIG. 9 is a schematic perspective view of the device shown in FIG. 1 arranged in a preliminary looped configuration in which the locking teeth are yet to be engaged.

### DETAILED DESCRIPTION

A preferred embodiment of the present invention will now be described with reference to the company figures. Reference numerals have been included in the following text to help identify the corresponding features in the figures. However in order to maintain clarity of the figures, not all of the reference numerals have been included in each figure.

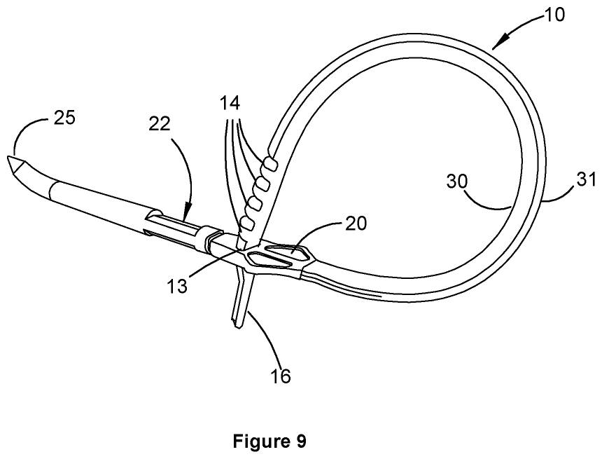

With reference to the figures, the medical device 10 has an elongated body including a stiffened portion 11 and a flexible portion 12, the stiffened portion 11 including a core or web 26 (best seen in FIG. 7) of the flexible material extending from the flexible portion 12 which integrally connects at least part of the stiffened portion 11 to the flexible portion 12. During installation of the medical device 10 in a patient, the stiffened portion 11 may be grasped with a pair of grasper or forceps and manoeuvred by a clinician between the internal organs as desired. The flexible portion 12 can then be manipulated and secured in a looped configuration, as shown in FIG. 9, about a gastric conduit. Once the medical device 10 has been implanted in position in the patient, the stiffened portion 11 is then severed from the device 10.

The device 10 can be manoeuvred between internal organs of a patient by grasping either one or both of the stiffened portion 11 or the flexible portion 12. One advantage of the device 10 is that the elongate body is ideally free of protrusions or projections, thereby allowing the device 10 to be manoeuvred into position using either the stiffened section 11 or a leading section 15 of the flexible portion 12 as the forward end of the device whilst being manoeuvred into position.

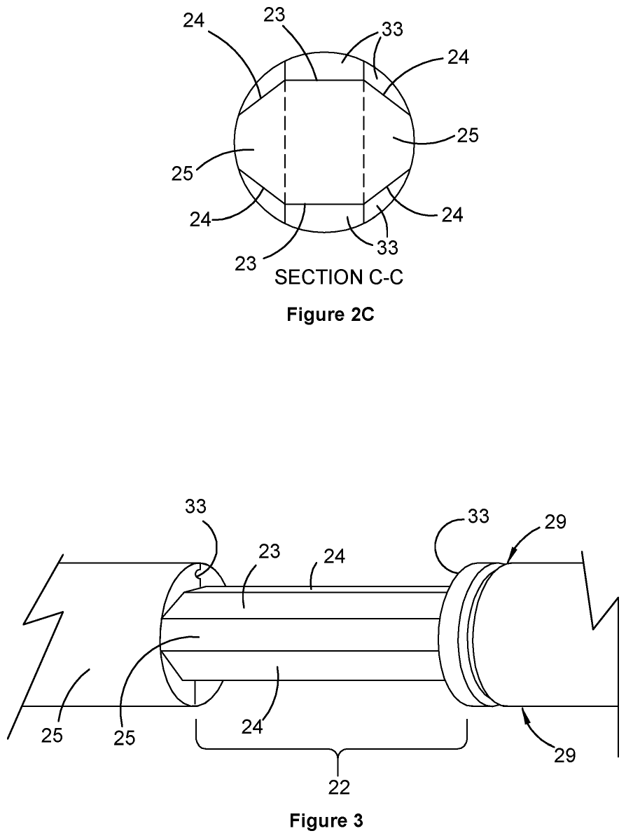

The device includes a grasping section 22, best be seen in FIGS. 2, 2C, 3 and 4, that is specifically configured to enhance the grip of jaws of a grasper or forceps to assist in manoeuvring the device into an implanted position. The grasping section 22 is configured to include a first pair of flat planar surfaces 23 that form part of a central web 26, best seen in FIG. 7, the central web 26 extending from the flexible portion 11 so as to form part of the stiffened portion 11. Although the surfaces 23 do not form part of the loop configuration, the surfaces 23 can at least in part align with inwardly facing and outwardly facing surfaces of the device 10. The flat planar surfaces 23 can be readily engaged by jaws that close in a parallel orientation relative to each other, for example, jaws that are pivotally mounted on spaced axes. The grasping section 22 also includes a second pair of gripping surfaces that have side planar surfaces 24 arranged at an angle to the first pair of gripping surfaces 23. As best seen FIG. 2C, the grasping section 22 includes a total four side surfaces 24, or in other words, two pairs of the side surfaces 24. The side planar surfaces 24 are form by the outer rigid members 25 of the stiffened portion 11, best seen in FIGS. 4 and 8.

The gripping section 22 is recessed below an outer perimeter of the stiffened portion 11 and abutment surfaces 33, best seen in FIG. 2C, extending laterally outwardly from the planar gripping surfaces 23 which can help a clinician to readily identify the gripping section 22. Moreover, the abutments surfaces 33 can engage the sides of jaws of a grasper or forceps to help prevent the stiffened portion 11 from pivoting or slipping out of the jaws. The abutment surfaces 33 may be provided at opposite ends of the gripping section. Ideally, the gripping section 22 is located adjacent to the flexible portion 12.

The device 10 includes a locking mechanism that is operable to secure the flexible portion 12 in the looped configuration at a selectable diameter about the gastric conduit. The locking mechanism includes an arch-shaped aperture 13 formed in a widest part of the body of the flexible portion 12 located adjacent to the stiffened portion 11, best seen in FIGS. 4 and 6, and teeth 14 that can engage the aperture 13.

The aperture 13 has an enlarged opening denoted by the line Y-Y in FIG. 6 on the inner surface of the loop configuration, compared to a reduced opening denoted by the line X-X on the outer surface of the loop configuration. The relative sizes of the enlarged and reduced openings, increases friction between teeth 14 and the aperture 13 as the teeth 14 move through the aperture 13. In one example, the line Y-Y may have a dimension of approximately 3 to 3.5 mm, and the line X-X may have a dimension of approximately 2 to 2.5 mm. The locking teeth 14 form part of the flexible portion 12 and are located remotely from the aperture 13 and adjacent to the leading section or a free end 15 of the flexible portion 12.

The locking teeth 14 are located adjacent to the free end 15 and remotely from the aperture 13. The length of the flexible portion 12 between the aperture 13 and the closest tooth of the locking teeth 14 to the aperture 13 may for example, be in the range of 50.0 to 100.0 mm. Ideally, the length of the flexible portion between the aperture 13 and the closest locking tooth is in the range of 60.0 to 80.0 mm.

The locking teeth 14 may be distributed over any suitable length and suitably over a length ranging from 10.0 to 30.0 mm, and suitably in the range of the 15.0 to 25.0 mm.

In order to close or form the looped configuration as shown in FIG. 9, the free end 15 of the flexible portion 12 is advanced through the aperture 13 such that at least one of the locking teeth 14 is pushed through the aperture 13. The diameter of the looped configuration can be adjusted by pulling locking teeth 14 through the aperture 13 as required. Specifically, in order to reduce the diameter, the free end 15 can be pulled to cause more locking teeth 14 to pass through the aperture 13. In order to increase the diameter, a clinician can use graspers to force the locking teeth 14 in a reverse direction through the aperture 13, this is expected to require a level of distortion of the locking teeth 14 and/or aperture 13 to increase diameter of the looped configuration.

The free end 15 of the device 10 has a nose 16 that can be threaded through the aperture 13 without engaging the aperture 13, and an engaging section 17 between the nose 16 and the locking teeth 14 for frictionally engaging the aperture 13. The engaging section 17 may have a cross-section or a notch 18 that is sized to engage the aperture 13, and thereby enable the device 10 to be arranged and held in a preliminary looped configuration prior to engaging locking teeth 14. This enables a clinician to re-position a grasper or forceps hold on the device 10 after initially threading the free end 15 through the aperture 13 and prior to engaging the locking teeth 14 to select the required diameter of the looped configuration.

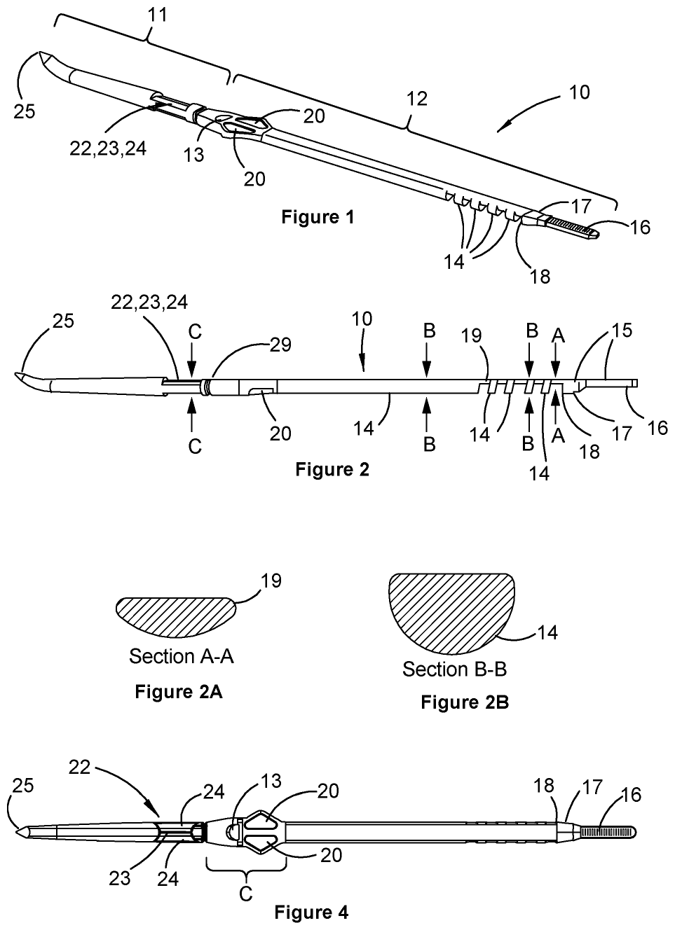

The nose 16 has a flattened profile, best seen in FIGS. 1 and 2, and a series of gripping grooves extending laterally to a longitudinal axis of the elongate body of the device 10. The grooves assist in gripping the nose 16 and passing the nose 16 through the aperture 13.

If desired, a clinician can also grasp the nose 16 to position the device 10 in an operative position about a patient's organs. In this instance the nose 16 can be used as the leading end for guiding the device 10 between organs and through connective tissue.

Ideally the device 10 has in the range of 4 to 6 locking teeth 14 which enables the device 10 to be adjusted over a range of diameters. The locking teeth 14 are separated by gaps that are defined by neck sections 19 in the flexible portion through cross-section A-A in FIG. 2, which is shown in detail in FIG. 2A. The locking teeth 14 have a cross-section B-B, which is shown in detail in FIG. 2B. The cross-section B-B also represents the constant cross-section along the length of the flexible portion 12 between the locking teeth 14 and suture openings 18.

As can best be seen in FIGS. 1 and 2B, the locking teeth 14 and the neck sections 19 defining the gaps between the locking teeth 14 have the same width, or a similar width, in a direction lateral to the longitudinal axis of the device 10. The locking teeth 14 do not protrude laterally in a width wise direction beyond the neck sections 19. Specifically, as can be seen in FIGS. 2A and 2B, the inner perimeter or faces of the neck 19 and the locking teeth 14 have the same width, which is also the same as the width as the remainder of the flexible portion 12. However as can be seen in FIG. 2, the locking teeth 14 extend beyond the neck portions 19 outward toward the outer diameter of the device 10. In other words, an outer peripheral section of the locking teeth 14 engages the aperture 13. As such the outer peripheral section of the locking teeth 14 is primarily responsible for engaging the aperture 13 and for releasably securing the device 10 in the looped configuration. This enables the loop configuration to be held in a ring of fixed diameter by the outer plane of the locking teeth 14 engaging an outer face of the aperture 13. In addition, it also allows the size of the ring to be increased by disengaging the plane in which locking teeth 14 engage the aperture 13.

Not only does engagement between the outer peripheral section of the locking teeth 14 provide secure engagement between the locking teeth 14 and the aperture 13, but in addition, this engagement allows a clinician some opportunity to safely increase the diameter of the looped configuration by distorting the teeth 14 and/or aperture 13 in the event that the looped configuration is over tightened.

In addition, the relative flexibility along the length of the flexible 12, is such that the loop configuration can form a circle of even diameter.

The suture openings 20 are also arranged in the widened part C of the flexible portion 12 for suturing the device 10 in the implanted position. The suture openings 20 are contained within elongated body of the device 10, and as shown in FIG. 6 do not protrude from the elongate body, particularly when the device is under tension, for example, while the device is being moved into position. The suture openings 20 are defined between outer walls 21 of the elongate body that are flexible and shortened relative in height than the thickness of the flexible portion 12.

FIGS. 1 and 4 illustrate the outer walls 21 as protruding outside of the elongate body 12, and FIG. 6 illustrates shows the outer walls 21 in alignment with the elongated body 12. The outer walls 21 can move from the position shown in FIGS. 1 and 4, to the position shown in FIG. 6 by the device being placed under tension. The outer walls 21 can also flex inwardly without being tensioned, for instance, the outer wall 21 can flex inwardly on contacting tissue when the device is being manoeuvred into position by a clinician. The flexibility of the outer walls 21 helps to reduce the likelihood of the outer walls 21 from forming an obstruction whilst the device 10 is being manoeuvred into an implant position. This provides an advantage of allowing the medical device 10 to be threaded between a patient's organs with a minimal risk of the medical device causing damage to surrounding organs and blood vessels.

It is also possible that the outer walls 21 may be located in the aligned position shown in FIG. 6, when in a relaxed state, that is, without tension or without external forces being applied to the outer walls 21 or to the device 10.

The stiffened portion 11 forms a trocar and has a curved tip 25 to help penetrate interconnective tissue as the device 10 is being manoeuvred into position.

The stiffened portion 11 of the device 10 includes a central web 26 that is integrally formed with the flexible portion 12. As can be seen in FIG. 7, the very end of the curved tip 25 is formed by the central web 26 to provide a softened extremity, and the tip 25 is supported by the outer rigid members 25 that are spaced from the very end of the tip 25 and extend longitudinally along the central web 26. As can be seen in FIGS. 8 and 9, the central web 26 includes a series of openings 28 and the outer rigid members 25 include corresponding lugs 27 that are positioned within the openings 28 to locate the outer rigid members 25. The outer rigid members 25 may also be attached to the central web 26 by any suitable means including chemical bonding or physical bonding. Suitably, the outer rigid members 26 and the central web 26 may be physically bonded.

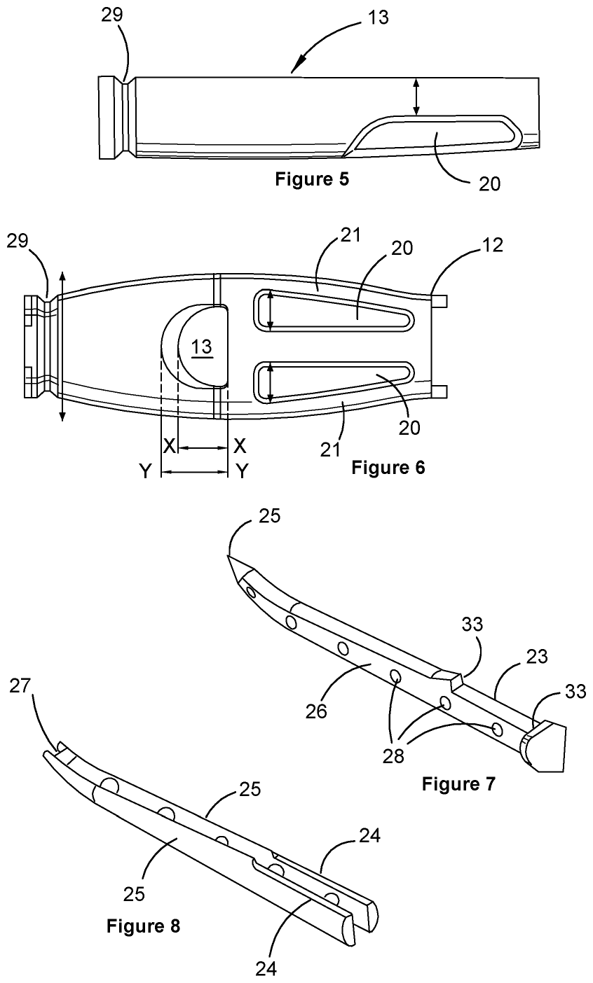

Ideally, the outer rigid member 25 and flexible material forming the web 26 and the flexible portion 12 may be made of any suitable biocompatible material. For example, the outer rigid member 25 may be made of polycarbonate or cross-linked polyethylene. The flexible portion 12 and web 26 may be made of a flexible elastomer such as a silicone rubber. During manufacture, the flexible portion 12 and web 26 can be over moulded into the outer rigid member 25. As can best be seen in FIGS. 5 and 6, the device 10 includes an annual groove 29 at the junction between the flexible portion 12 and the stiffened portion 11 that denotes a suitable location for severing the stiffened portion 11 once the device has been located in an implant position.

The flexible portion 12 of the elongated body has an asymmetric cross-section, which is suitably hemispherical or arched, such as cross-section B-B in FIG. 3B, which can enable a clinician to identify when the flexible portion has been inadvertently twisted about its longitudinal axis during the medical procedure. Specifically, when the device is located in a looped configuration, the upwardly facing surface of the device shown in FIGS. 1 and 4 has a flat profile, and forms an inner diameter 30 of the closed loop configuration seen in FIG. 9. The downwardly facing surface of the device 10 shown in FIG. 1 has an arched profile and forms the outer diameter 31 of the looped configuration.

Those skilled in the art of the present invention will appreciate that many variations and modifications may be made to the preferred embodiment described herein without departing from the spirit and scope of the present invention.

1. A medical device that can be implanted, the medical device comprising:
an elongate body having a flexible portion and a stiffened portion extending from the flexible portion, the stiffened portion can be gripped by a grasper and thereby manoeuvre the stiffened portion between internal organs of a patient to place the device about a gastric conduit of a patient;
a locking mechanism that is operable to secure the flexible portion in a looped configuration at a selectable diameter about the gastric conduit, the locking mechanism having: i) an aperture in the flexible portion that is positioned adjacently to the stiffened portion, and ii) a plurality of locking teeth that form part of the flexible portion that are positioned remotely from the stiffened portion;
wherein the flexible portion includes a free end that can be advanced through the aperture until one of the locking teeth engages the aperture and secures the looped configuration in the desired diameter.

2. The medical device according to claim 1, wherein the device has a grasping section having a gripping surface for jaws of a grasper, the gripping surface being adapted to receive the jaws of a grasper by being recessed to an outer face of the body.

3. The medical device according to claim 2, wherein a recessed area extends about at least part of the perimeter and has a smaller cross-section than an adjacent section of the stiffened portion, and the grasping section includes an at least one abutment surface that extends laterally to the gripping surfaces and are configured to allow jaws of the grasper or forceps to butt against the abutment surface.

4. The medical device according to claim 3, wherein the grasping section has two abutment surfaces that extend outward at opposite ends of the recessed area.

5. The medical device according to claim 2, wherein the grasping section is located in the stiffened portion only.

6. (canceled)

7. The medical device according to claim 2, wherein the gripping surfaces of the grasping section have flat planar surfaces on which the jaws of a grasper can contact when closing, and wherein the flat planar surfaces are arranged about at least part of a perimeter of the recessed area of the stiffened portion and includes a first pair of gripping surfaces that face outwardly from opposite sides of the body of the device.

8. (canceled)

9. The medical device according to claim 7, wherein the gripping section has at least one set of second pair of gripping surfaces having side planar surfaces arranged at an angle to the first pair of planar surfaces, the second pair of gripping surfaces are oppositely disposed to each other about the body of the device and face outwardly of the body of the device.

10. (canceled)

11. (canceled)

12. (canceled)

13. The medical device according to claim 1, wherein the device includes at least one suture opening for suturing the device in an operative position when implanted in a patient, the suture opening being arranged within the elongate body of the flexible portion.

14. The medical device according to claim 13, wherein the device has two suture openings located side-by-side in the elongated body of the device, and each suture opening is defined between an inner wall and one of two outer walls of the elongate body.

15. (canceled)

16. (canceled)

17. (canceled)

18. The medical device according to claim 14, wherein the outer wall of the or each suture opening includes a flexible part, and when in a relaxed state the flexible part aligns with the elongated body and can move inward and/or outward.

19. (canceled)

20. (canceled)

21. The medical device according to claim 14, wherein the free end of the device has a reduced cross-section portion that can be threaded through the aperture without frictionally engaging the aperture.

22. The medical device according to claim 21, wherein the free end has an engaging section extending from the reduced cross-section portion, the engaging section having an enlarged cross-section that is sized to frictionally engage the aperture to retain the device in the preliminary looped configuration without engaging the locking teeth.

23. (canceled)

24. The medical device according to claim 22, wherein the reduced cross section has a flattened profile that can be engaged by jaws of a grasper and has ridges extending transversely to a longitudinal axis of the device to assist in the jaws gripping onto the leading section.

25. (canceled)

26. (canceled)

27. The medical device according to claim 1, wherein at least part of the stiffened portion is integrally formed with the flexible portion, and wherein the stiffened portion has a centrally located web that is integrally formed with the flexible portion, the centralised web extends diametrically across the stiffened portion, and wherein the stiffened portion includes an outer rigid portion having opposite outer side flanks extending in a direction along the stiffened portion, and wherein the outer side flanks are interconnected by lugs that extend through the central web.

28. (canceled)

29. (canceled)

30. (canceled)

31. The medical device according to claim 1, wherein the flexible portion has a shaped cross-section in which an inwardly facing surface of the elongated body has a flattened profile and the outwardly facing surface of the elongate body has a curved profile.

32. The medical device according to claim 31, wherein the shaped cross-section extends along the entire length of flexible portion between the aperture to locking teeth.

33. The medical device according to claim 1, wherein the aperture of the locking mechanism is disposed adjacent to the stiffened portion.

34. The medical device according to claim 1, wherein the aperture has a first opening on the inwardly facing surface of the device, when in a looped configuration, that is larger than a second opening on at an outwardly facing of the device.

35. (canceled)

36. The medical device according to claim 1, wherein the locking teeth of the flexible portion are separated by neck sections, and the neck sections and the locking teeth both have the same or a similar width at an inwardly facing surface of the device.

37. A medical device that can be implanted, the medical device comprising:
an elongate body having a flexible portion and a stiffened portion extending from the flexible portion, the stiffened portion can be gripped by a grasper and thereby manoeuvre the stiffened portion between internal organs of a patient to place the device about a gastric conduit of a patient;
a locking mechanism that is operable to secure the flexible portion in a looped configuration at a selectable diameter about the gastric conduit, the locking mechanism having: i) an aperture in the flexible portion that is positioned adjacently to the stiffened portion, and ii) a plurality of locking teeth that form part of the flexible portion that are positioned remotely from the stiffened portion;
a grasping section having a gripping surface for jaws of a grasper, the gripping surface being adapted to receive the jaws of a grasper by being recessed to an outer face of the body;
a recessed area extends about at least part of the perimeter and has a smaller cross-section than an adjacent section of the stiffened portion, and the grasping section includes an at least one abutment surface that extends laterally to the gripping surfaces and are configured to allow jaws of the grasper or forceps to butt against the abutment surface;
wherein the flexible portion includes a free end that can be advanced through the aperture until one of the locking teeth engages the aperture and secures the looped configuration in the desired diameter;
wherein the device includes at least one suture opening for suturing the device in an operative position when implanted in a patient, the suture opening being arranged within the elongate body of the flexible portion; and
wherein the flexible portion has a shaped cross-section in which an inwardly facing surface of the elongated body has a flattened profile and the outwardly facing surface of the elongate body has a curved profile.
